(19) United States
(12) Patent Application Publication
(10) Pub. No.: US 2025/0213177 A1
(43) Pub. Date: Jul. 3, 2025

(54) SEIZURE DETECTION SYSTEM AND METHOD THEREOF

(71) Applicant: Shikhar KHURANA, Bangalore (IN)

(72) Inventor: Shikhar KHURANA, Bangalore (IN)

(21) Appl. No.: 18/844,045

(22) PCT Filed: Mar. 4, 2023
(86) PCT No.: PCT/IB2023/052033

§ 371 Date: Sep. 4, 2024

(30) Foreign Application Priority Data
Mar. 4, 2022 (IN) 202241011875

(51) Int. Cl. A61B 5/00; A61B 5/0205; A61B 5/021; A61B 5/024; A61B 5/31; A61B 5/372; G16H 50/30

(52) U.S. Cl. CPC A61B 5/4094; A61B 5/0205; A61B 5/021; A61B 5/02405; A61B 5/02416; A61B 5/31; A61B 5/372; A61B 5/7203; A61B 5/7264; A61B 5/746; G16H 50/30

(57) ABSTRACT

Disclosed is a seizure detection system (100) including an input unit (104), a pre-processing unit (106), first processing circuitry (108), a post-processing unit (110), second processing circuitry (112), and an output unit (114). The input unit (104) obtains electroencephalography (EEG) data and photoplethysmography (PPG) data to generate a first signal representing the EEG data and the PPG data. The pre-processing unit (106) pre-processes the PPG and the EEG data to generate a second signal. The first processing circuitry (108) generate three outputs, a combined output, and a third signal based on the combined output. The post-processing unit (110) generate an event score and obtain one or more physiological parameters of a subject. The second processing circuitry (112) combines the event score and the one or more physiological parameters to generate a fourth signal representing a final output. The output unit (114) predicts a seizure based on the final output.

### TECHNICAL FIELD

The present disclosure relates generally to neurological disorders. More particularly, the present disclosure relates to seizure detection system and method thereof.

### BACKGROUND

Epilepsy is one of the most common chronic neurological disorders and is defined as a person having at least two unprovoked seizures. People suffering from epilepsy tend to have more cognitive and physical problems (such as Memory impairment, ADHD (attention deficit hyperactivity disorder), autism, accidental injuries, fractures and bruising from injuries related to seizures), as well as higher rates of psychological conditions, including anxiety and depression. Similarly, the risk of premature death in people with epilepsy is up to three times higher than in the general population According to the World Health Organization, more than 50 million people worldwide have epilepsy and nearly 80% of them live in low-and middle-income countries. An estimated 70% of people with epilepsy could be seizure-free if timely and scientifically diagnosed and treated, which would require early and accurate detection of seizures that can help people suffering from epilepsy to get early medical attention and lead a better life.

Currently, seizure detection and classification are an uphill task requiring manual annotations and inputs from highly skilled physicians. There have been many studies and systems designed for seizure detection which apply deep learning techniques to electroencephalogram (EEG) brain signals to predict the onset of seizures. But such algorithms have not been effective enough to be clinically deployed, because of the low sensitivity of the seizure event detection and the dearth of relevant EEG data with representative seizure cases and patient samples.

Contemporary attempts at seizure detection using only scalp recordings have had high false alarm rates. This stems from the basis of various noise signals and artefacts that are invariably produced because of eye movements, muscle movement, shivering or chewing, voluntary movements by patient and background or equipment generated noise. Conversely, studies on seizure detection which use parameters relating to autonomic changes, such as heart rate variability (HRV), blood pressure, body temperature etc., are low.

Therefore, there exists a need for a method of prediction and detection of seizures that is free of the above-mentioned limitations and can provide more accurate results.

### SUMMARY

In view of the foregoing, a seizure detection system is disclosed. The seizure detection system includes an input unit configured to obtain electroencephalography (EEG) data and photoplethysmography (PPG) data to generate a first signal representing the EEG data and the PPG data. The seizure detection system further includes a pre-processing unit coupled to the input unit, and configured to, upon receipt of the first signal, pre-process the PPG data and the EEG data to generate a second signal. The seizure detection system further includes first processing circuitry coupled to the pre-processing unit, and configured to, upon receipt of the second signal, (i) generate at least three outputs and (ii) generate a combined output by combining the at least three outputs. The first processing circuitry further generates a third signal based on the combined output. The seizure detection system further includes a post-processing unit coupled to the first processing circuitry, and configured to, upon receipt of the third signal (i) post-process the combined output to generate an event score and (ii) compute one of, the EEG data and the PPG data to obtain one or more physiological parameters of a subject. The seizure detection system further includes second processing circuitry coupled to the post-processing unit, and configured to combine the event score and the one or more physiological parameters to generate a fourth signal representing a final output. The seizure detection system further includes an output unit coupled to the second processing circuitry, and configured to, upon receipt of the fourth signal, predict a seizure based on the final output.

In some embodiments, the seizure detection system further includes a database that is coupled to the input unit such that the database stores the EEG data and PPG data, wherein the input unit is configured to obtain the EEG data and the PPG data from the database.

In some embodiments, the input unit includes a plurality of sensors that are configured to sense EEG and PPG signals representing the EEG and the PPG data, respectively.

In some embodiments, to pre-process the EEG data, the pre-processing unit is configured to remove a plurality of artefacts from the EEG data.

In some embodiments, the output unit comprising third processing circuitry that is coupled to the second processing circuitry, and configured to (i) predict the seizure and (ii) determine, upon receipt of the fourth signal, phase of the seizure.

In some embodiments, the third processing circuitry is further configured to generate an alert signal upon prediction of the seizure.

In some embodiments, the seizure detection system further includes a notifier that is configured to generate a notification pertaining to the seizure upon generation of the alert signal to notify at least one of, a care-giver, a health-personnel, and a health authority.

In some embodiments, the one or more physiological parameters comprises heart rate variability and blood pressure.

In some embodiments, the third processing circuitry is further configured to generate a null signal, when at least one of (i) seizure events<30 seconds, (ii) seizure events<15 seconds, and (iii) merged seizure events with P (seizure)<82%, is met.

In some aspects, a method for detecting seizure is disclosed. The method includes obtaining, by way of an input unit, electroencephalography (EEG) data and photoplethysmography (PPG) data, to generate a first signal representing the EEG data and the PPG data. The method further includes pre-processing, by way of a pre-processing unit that is coupled to the input unit, the EEG data, upon receipt of the first signal, to generate the second signal. The method further includes generating, by way of first processing circuitry that is coupled to the pre-processing unit, upon receipt of the second signal, at least three outputs. The method further includes generating, by way of the first processing circuitry, a combined output by combining the at least three outputs to generate a third signal based on the combined output. The method further includes post-processing, by way of a post-processing unit that is coupled to the first processing circuitry, upon receipt of the third signal, the combined output to generate an event score. The method further includes computing, by way of the post-processing unit, one of, the EEG data and the PPG data to obtain one or more physiological parameters of a subject. The method further includes combining, by way of second processing circuitry that is coupled to the post-processing unit, the event score and the one or more physiological parameters to generate a fourth signal representing a final output. The method further includes predicting, by way of an output unit that is coupled to the second processing circuitry, upon receipt of the fourth signal, a seizure based on the final output.

In some embodiments, the method further includes alerting, by way of a notifier of the output unit, the subject about the seizure, wherein to alert, the output unit is configured to generate a notification pertaining to the seizure upon generation of the alert signal to notify at least one of, a care-giver, a health-personnel, and a health authority.

### BRIEF DESCRIPTION OF DRAWINGS

The above and still further features and advantages of embodiments of the present disclosure becomes apparent upon consideration of the following detailed description of embodiments thereof, especially when taken in conjunction with the accompanying drawings, and wherein:

FIG. 1 illustrates a block diagram of a seizure detection system, in accordance with an embodiment herein;

FIG. 2A illustrates a graphical representation of pulse tracing, in accordance with an embodiment herein;

FIG. 2B illustrates a graphical representation of morphology of two PPG waves, in accordance with an embodiment herein; and

FIG. 3 illustrates a flow-chart of a method for detecting the seizure, in accordance with an embodiment herein.

To facilitate understanding, like reference numerals have been used, where possible, to designate like elements common to the figures.

### DETAILED DESCRIPTION

Various embodiments of the present disclosure provide a seizure detection system and method thereof. The following description provides specific details of certain embodiments of the disclosure illustrated in the drawings to provide a thorough understanding of those embodiments. It should be recognized, however, that the present disclosure can be reflected in additional embodiments and the disclosure may be practiced without some of the details in the following description.

The various embodiments including the example embodiments are now described more fully with reference to the accompanying drawings, in which the various embodiments of the disclosure are shown. The disclosure may, however, be embodied in different forms and should not be construed as limited to the embodiments set forth herein. Rather, these embodiments are provided so that this disclosure is thorough and complete, and fully conveys the scope of the disclosure to those skilled in the art. In the drawings, the sizes of components may be exaggerated for clarity.

It is understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

The subject matter of example embodiments, as disclosed herein, is described specifically to meet statutory requirements. However, the description itself is not intended to limit the scope of this disclosure. Rather, the inventor/inventors have contemplated that the claimed subject matter might also be embodied in other ways, to include different features or combinations of features similar to the ones described in this document, in conjunction with other technologies. Generally, the various embodiments including the example embodiments relate to seizure detection system and method thereof.

As mentioned, there remains a need for an efficient system and method to predict and detect seizures with high accuracy and minimum number of false positive results. Accordingly, the present disclosure provides a seizure detection system that detects the seizures with high accuracy by considering electroencephalography data and other autonomous factors of the subject such as heart rate variability (HRV) and blood pressure of the subject.

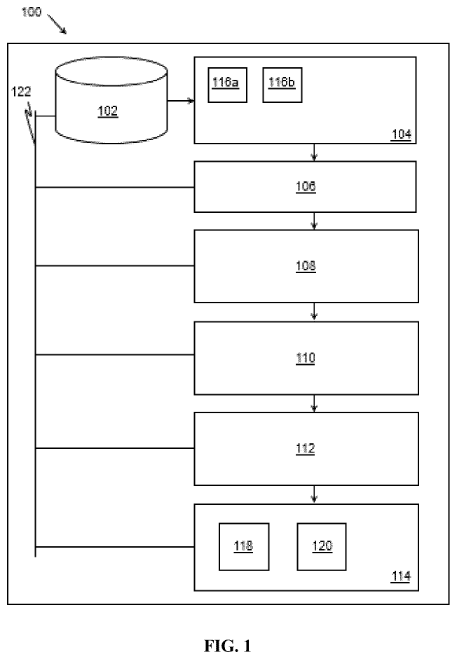

FIG. 1 illustrates a block diagram of a seizure detection system 100, in accordance with an embodiment herein. The seizure detection system 100 may predict a seizure in a subject. The seizure detection system 100 may further predict phase of the seizure in the subject. The seizure detection system 100 may asses changes in autonomic parameters surrounding an impeding seizure attack.

In some embodiments, the seizure detection system 100 may be used to determine epilepsy of the subject.

The seizure detection system 100 may include a database 102, an input unit 104, a pre-processing unit 106, first processing circuitry 108, a post-processing unit 110, second processing circuitry 112, and an output unit 114. The input unit 104 may include a plurality of sensors of which first and second sensors 116a and 116b are shown (hereinafter collectively referred to and designated as “the sensors 116”). The output unit 114 may include third processing circuitry 118 and a notifier 120.

In some embodiments, the database 102, the input unit 104, the pre-processing unit 106, the first processing circuitry 108, the post-processing unit 110, the second processing circuitry 112, and the output unit 114 may be coupled to each other by a communication network 122. The communication network 122 may be one of, a wired network and a wireless network.

In some embodiments, each of the first through third processing circuitries 108, 112, and 118 may be any or a combination of microprocessor, microcontroller, Arduino Uno, At mega 328, Raspberry Pi or other similar processing unit, and the like. In yet another embodiment, each of the first through third processing circuitries 108, 112, and 118 may include one or more processors coupled with a memory (not shown) such that the memory storing computer-readable instructions executable by the one or more processors.

In some embodiments, each of the first through third processing circuitries 108, 112, and 118 may be implemented as one or more microprocessors, microcomputers, microcontrollers, digital signal processors, central processing units, logic circuitries, and/or any devices that manipulate data based on operational instructions stored in a memory. The computer-readable instructions or routines stored in the memory may be fetched and executed to create or share the data units over a network service. The memory may include any non-transitory storage device including, for example, volatile memory such as RAM, or non-volatile memory such as EPROM, flash memory, and the like.

In some embodiments, each of the first through third processing circuitries 108, 112, and 118 may be implemented as a combination of hardware and programming (for example, programmable instructions) to implement one or more functionalities of each of the first through third processing circuitries 108, 112, and 118. In examples described herein, such combinations of hardware and programming may be implemented in several different ways. For example, the programming for each of the first through third processing circuitries 108, 112, and 118 may be processor executable instructions stored on a non-transitory machine-readable storage medium and the hardware for each of the first through third processing circuitries 108, 112, and 118 may include a processing resource (for example, one or more processors), to execute such instructions. In the present examples, the machine-readable storage medium may store instructions that, when executed by the processing resource, implement each of the first through third processing circuitries 108, 112, and 118. In such examples, each of the first through third processing circuitries 108, 112, and 118 may include the machine-readable storage medium storing the instructions and the processing resource to execute the instructions, or the machine-readable storage medium may be separate but accessible to each of the first through third processing circuitries 108, 112, and 118 and the processing resource. In other examples, each of the first through third processing circuitries 108, 112, and 118 may be implemented by an electronic circuitry.

The database 102 may be configured to store electroencephalography (EEG) data and photoplethysmography (PPG) data of the subject. In some embodiments of the present disclosure, the database 102 may be a Temple university hospital database. The Temple university hospital database may include world's largest publicly available clinical EEG data. The Temple university hospital database may be a database with over 300 subjects and spanning 280 sessions that may include seizures.

The input unit 104 may be coupled to the database 102. The input unit 104 may include a suitable logic circuitry that may be configured to perform one or more operations, for example, to obtain the EEG data and the PPG data from the database 102. The input unit 104 may further be configured to obtain the EEG data and the PPG data from the subject. The input unit 104 may further be configured to generate a first signal such that the first signal represents the EEG data and the PPG data. The sensors 116 may be disposed on the body of the subject to obtain the EEG data and the PPG data from the subject. Specifically, the sensors 116 may be configured to sense EEG and PPG signals that may represent the EEG data and the PPG data, respectively.

In some embodiments, the sensors 116 may be disposed on the scalp of the subject in order to sense EEG signals.

In some embodiments, the input unit 104 may include, but not limited to, an electrode, an optical device, an imaging device, and the like. Embodiments of the present disclosure are intended to include and/or otherwise cover any kind of input unit 104 that may be able to obtain the EEG data and the PPG data from the subject.

In some embodiments, the input unit 104 may include a machine learning (ML) engine that may facilitate the input unit 104 to obtain the EEG data and the PPG data. Specifically, the ML engine may be convoluted neural network.

In some embodiments, the sensors 116 may be a plurality of gyroscope sensors. The plurality of gyroscopic sensors may be configured to differentiate heart rate change associated with the subject. Specifically, the plurality of gyroscope sensors may be configured to differentiate between movement induced heart rate change and seizure related heart rate change.

The pre-processing unit 106 may be coupled to the input unit 104. The input unit 104 may be further configured to transmit the first signal to the pre-processing unit 106. The pre-processing unit 106 may be configured to, upon receipt of the first signal, pre-process the EEG data to generate a second signal. Specifically, the pre-processing unit 106, to pre-process the EEG data, may be configured to remove a plurality of artefacts from the EEG data.

In some exemplary embodiments, the pre-processing unit 106 may be configured to pre-process the EEG data by three different procedures or pipelines.

Specifically, the pre-processing unit 106 may remove the plurality of artefacts from the EEG data by three different procedures or pipelines. Firstly, input raw EEG data may be used. The raw EEG data may be un-processed data i.e., without processing. The second pipeline may require a Multichannel Wiener filter. The Multichannel Wiener filter may be semi-automated, as the plurality of artefacts may be manually annotated and then a covariance matrix may be constructed. The Multichannel Wiener filter may be used for auto-artifact identification (max SNR filter), clustering (using K-means), and removing the identified artifact. The third pipeline may use Independent Component Analysis (ICA) via a neural network, IClabel (model), and the best classifier using EEG Lab (an open-source environment for human EEG and signal data analysis and processing) that may be trained on 6000 EEGs and compiled over 20 k independent components. The third pipeline may identify and cluster signals according to source. Specifically, the third pipeline may retain signal corresponding to Brain data and eliminate signals corresponding to Muscle, Eyes, and Line noise.

The first processing circuitry 108 may be coupled to the pre-processing unit 106. The pre-processing unit 106 may be further configured to transmit the second signal to the first processing circuitry 108. The first processing circuitry 108 may be configured to generate at least three outputs obtained from the three different pre-processing pipelines. The first processing circuitry 108 may further be configured to generate a combined output by combining the at least three outputs. The first processing circuitry 108 may further be configured to generate a third signal based on the combined output.

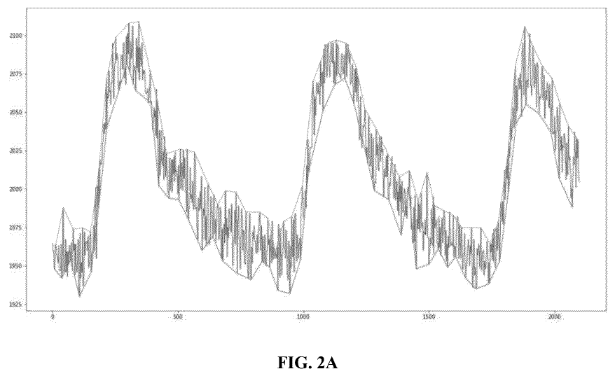

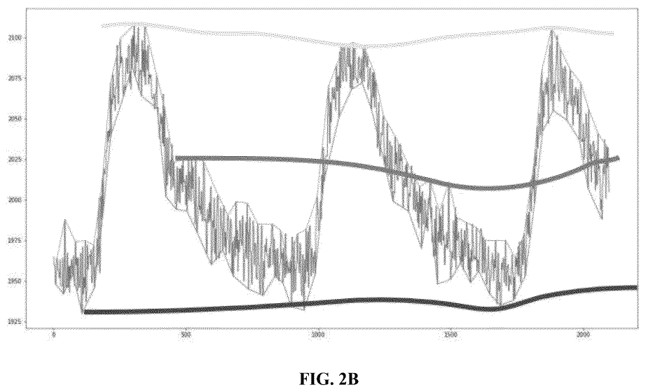

The post-processing unit 110 may be coupled to the first processing circuitry 108. The first processing circuitry 108 may be configured to transmit the third signal to the post-processing unit 110. The post-processing unit 110 may include a suitable logic circuitry that may be configured to perform one or more operations, for example, to post process the combined output to generate an event score upon receipt of the third signal. The event score may eliminate the risk of errors while predicting seizures. The event score may be interpreted by a health professional or a doctor to eliminate the risk of errors. The post-processing unit 110 may further be configured to compute one of, the EEG data and the PPG data to obtain one or more physiological parameters of the subject. For example, the one or more physiological parameters may include, but not limited to, heart rate variability (HRV), blood pressure (BP), and other autonomic factors associated with the subject. In some exemplary embodiments, the post-processing unit 110 may compute the PPG data by applying single pulse envelope wave (SPEV), pulse tracing envelope wave (PTEV), and kernel density estimates (KDE) (as explained in detail in FIG. 2A and FIG. 2B).

FIG. 2A illustrates a graphical representation of pulse tracing, in accordance with an embodiment herein. FIG. 2B illustrates a graphical representation of morphology of two PPG waves, in accordance with an embodiment herein. Specifically, FIG. 2A depicts a blue thin serrated line for pulse tracing, yellow thick line, and red thick line for/depicting Pulse tracing envelope wave (PTEV). The thick blue line is a notch tracing line, while orange thin line and green thin line encompass single pulse envelope wave (SPEV).

The post-processing unit 110 may compute the PPG data by acquiring the PPG data in the form of peak(s) representing pulse(s) on X-axis and Y-axis. The post-processing unit 110 may then apply single pulse envelope wave (SPEV) and pulse tracing envelope wave (PTEV) to compute median and standard deviation of height of all comparable points of the pulse. The post-processing unit 110 may further compute median and standard deviation of height of all comparable points of the various envelope waves to identify start and end points of an individual pulse cycle. The post-processing unit 110 may further break the input sample into individual pulses to compute mean height of all points in each of the individual pulses. The post-processing unit 110 may further procure a plurality of the summed points from all of the obtained envelope waves and continue thereof for each of the individual pulses. The post-processing unit 110 may then receive summated statistics that predicts physiological state of the subject by detecting divergence between predicted and actual statistics.

In some exemplary embodiments, the post-processing unit 110 may compute the PPG data by acquiring the PPG data in the form of peak(s) representing pulse(s) on the X-axis and the Y-axis. The post-processing unit 110 may normalize the Y-axis, compute median and standard deviation of height of all comparable points of the pulse. The post-processing unit 110 may compute median and standard deviation of height of all comparable points of the pulse and identify start and end points of an individual pulse cycle. The post-processing unit 110 may break the input sample into individual pulses to compute mean height of all points in each of the individual pulses. The post-processing unit 110 may validate, if mean of the individual pulses is out of a limit of the sample median with or without the standard deviation. The post-processing unit 110 may apply kernel density estimates (KDE) on each of the individual pulse and procures a plurality of the summed points from all kernels obtained from the KDE. The post-processing unit 110 may obtain mean KDE for all the individual pulses and receive a summated pulse. The post-processing unit 110 may include a suitable imaging device (not shown) that may acquire an image of the body of the subject by recording a video of skin for at least 5 seconds. The post-processing unit 110 may select the best second data for capturing a plurality of images of the skin of the subject. The post-processing unit 110 may convert thereof into pixel level data. The pixel level data may be a three dimensional data. The post-processing unit 110 may superimpose the images one above another to acquire a single image of two dimensions reflecting blood flow into the skin tissue. The post-processing unit 110 may compute pulse variability and mean. The post-processing unit 110 may replace temperature by the standard deviation and convert the analysis into color coded heat map. The post-processing unit 110 may track the fluctuating heat of depth of the surface of the skin. The post-processing unit 110 may compute width of the envelope of each pulse variation and capture a plurality of images of a target area using an infrared camera at a plurality of different time intervals. The post-processing unit 110 may add visible light to the images for differentiating the type of tissue and adding ultraviolet light to the images for determining infection of a tissue of the subject. The post-processing unit 110 may capture facial patterns of the subject by detecting, tracking, and recognizing thereof, and merging the pulse reading analysis with the imaging analysis and facial pattern analysis for detecting precise divergence between the predicted and actual data for predicting the clinical and non-clinical outcomes of the subject. The second processing circuitry 112 may be coupled to the post-processing unit 110. The second processing circuitry 112 may be configured to combine the event score and the one or more physiological parameters to generate a fourth signal. The fourth signal may represent a final output.

In some embodiments, the final output may be ratio of two physiological parameters. Specifically, the final output may be the ratio of sympathetic nerve signals to parasympathetic nerve signals.

In some embodiments, the final output may indicate probability of existence of the seizure in the subject.

The output unit 114 may be coupled to the second processing circuitry 112. Specifically, the third processing circuitry 118 may be coupled to the second processing circuitry 112. The second processing circuitry 112 may be configured to transmit the fourth signal to the output unit 114. Specifically, the second processing circuitry 112 may be configured to transmit the fourth signal to the third processing circuitry 118. The third processing circuitry 118 may be configured to predict the seizure upon receipt of the fourth signal. Specifically, the third processing circuitry 118 may predict the seizure based on the final output. The third processing circuitry 118 may further be configured to determine phase of the seizure upon receipt of the fourth signal. Specifically, the third processing circuitry 118 may differentiate between preictal and interictal phase of the seizure. The third processing circuitry 118 may further be configured to generate an alert signal upon prediction of the seizure. The notifier 120 may be configured to generate a notification pertaining to the seizure upon generation of the alert signal. Specifically, the notifier 120 may be configured to generate the notification for at least one of, a care-giver, a health-personnel, and a health authority. In some embodiments, the notifier 120 may be configured to generate the notification in a hand-held device or a wearable device carried by the at least one of, the care-giver and the health-personnel.

In some preferred embodiments, the post-processing unit 110 performs post-processing of the combined output by removal of EEG sessions that had:
Seizure events<30 seconds apart were merged
Seizure events<15 seconds were rejected
Merged seizure events with P (Seizure)<82% were rejected

The seizure detection system 100 may not give notification, if any of the three conditions (as mentioned hereinabove) is met. Specifically, the third processing circuitry 118 may be configured to generate a null signal, if any of the above-mentioned conditions for seizures is met. The null signal may prevent generation of notification by the notifier 120.

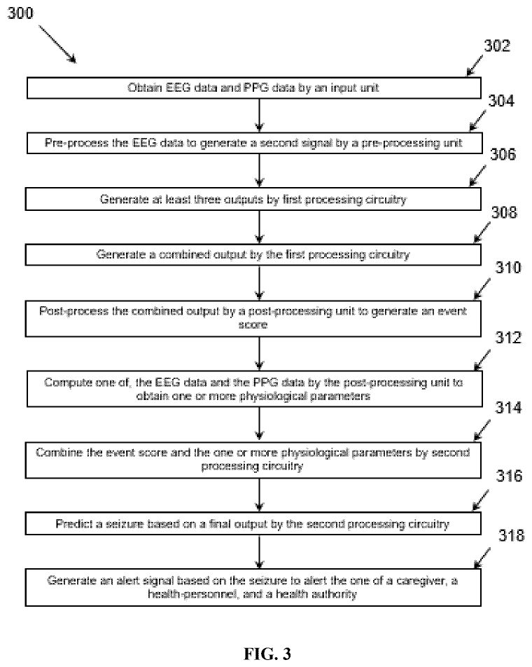

FIG. 3 illustrates a flow-chart of a method 300 for detecting the seizure, in accordance with an embodiment herein. The method 300 may include following steps for detecting the seizure:

At step 302, the seizure detection system 100, by way of the input unit 104, may obtain the EEG data and the PPG data to generate the first signal representing the EEG data and the PPG data. The input unit 104 may be coupled to the database 102. The input unit 104 may include the suitable logic circuitry that may be configured to perform the one or more operations, for example, to obtain the EEG data and the PPG data from the database 102. The sensors 116 may be disposed on the body of the subject. The sensors 116 may be configured to sense the EEG and PPG signals that may represent the EEG data and the PPG data, respectively.

At step 304, the seizure detection system 100, by way of the pre-processing unit 106 that may be coupled to the input unit 104, may pre-process the EEG data to generate the second signal. The input unit 104 may be further configured to transmit the first signal to the pre-processing unit 106. The pre-processing unit 106 may, upon receipt of the first signal, pre-process the EEG data to generate the second signal. Specifically, the pre-processing unit 106, to pre-process the EEG data, may be configured to remove the plurality of artefacts from the EEG data.

At step 306, the seizure detection system 100, by way of the first processing circuitry 108 that may be coupled to the pre-processing unit 106, may generate the at least three outputs upon receipt of the second signal. The pre-processing unit 106 may be further configured to transmit the second signal to the first processing circuitry 108. The first processing circuitry 108 may be configured to generate at least three outputs.

At step 308, the seizure detection system 100, by way of the first processing circuitry 108, may generate the combined output by combining the at least three outputs to generate the third signal based on the combined output. The first processing circuitry 108 may further be configured to generate the third signal based on the combined output.

At step 310, the seizure detection system 100, by way of the post-processing unit 110 that may be coupled to the first processing circuitry 108, may post-process the combined output to generate the event score upon receipt of the third signal. The first processing circuitry 108 may be configured to transmit the third signal to the post-processing unit 110.

At step 312, the seizure detection system 100, by way of the post-processing unit 110, may compute one of, the EEG data and the PPG data to obtain one or more physiological parameters of the subject. For example, the post-processing unit 110 may compute the PPG data to obtain heart rate variability (HRV), blood pressure (BP), and other autonomic factors associated with the subject.

At step 314, the seizure detection system 100, by way of the second processing circuitry 112 that may be coupled to the post-processing unit 110, may combine the event score and the one or more physiological parameters to generate the fourth signal. The fourth signal may represent the final output.

At step 316, the seizure detection system 100, by way of the output unit 114 that may be coupled to the second processing circuitry 112, may predict the seizure. Specifically, the output unit 114 may be configured to predict the seizure based on the final output. The second processing circuitry 112 may be configured to transmit the fourth signal to the output unit 114. Specifically, the second processing circuitry 112 may be configured to transmit the fourth signal to the third processing circuitry 118. The third processing circuitry 118 may be configured to predict the seizure upon receipt of the fourth signal. Specifically, the third processing circuitry 118 may predict the seizure based on the final output. The third processing circuitry 118 may further be configured to determine phase of the seizure upon receipt of the fourth signal.

At step 318, the seizure detection system 100, by way of the output unit 114, may generate the alert signal based on the seizure, to alert the care-giver, the health-personnel, and the health authority. The third processing circuitry 118 may further be configured to generate the alert signal upon prediction of the seizure. The notifier 120 may be configured to generate the notification pertaining to the seizure upon generation of the alert signal. Specifically, the notifier 120 may be configured to generate the notification for the at least one of, the care-giver, the health-personnel, and the health authority.

Thus, the seizure detection system 100 may provide following advantages that may be derived from the functional aspects of the seizure detection system 100:
The seizure detection system 100 accurately predicts the seizure and the phase of the seizure in the subject.
The seizure detection system 100 generates notification for the care-giver, the health-personnel, and the health authority, which ensures proper treatment of the subject (patient).
The seizure detection system 100 is able to detect early stage of seizure using interictal periods of EEG signals. The subject may immediately take medication to prevent further seizures.
The seizure detection system 100 uses machine learning model that reduces cost and time of epileptic seizure and EEG signal analysis while reducing the workload of doctors.

The foregoing discussion of the present disclosure has been presented for purposes of illustration and description. It is not intended to limit the present disclosure to the form or forms disclosed herein. In the foregoing Detailed Description, for example, various features of the present disclosure are grouped together in one or more embodiments, configurations, or embodiments for the purpose of streamlining the disclosure. The features of the embodiments, configurations, or embodiments may be combined in alternate embodiments, configurations, or embodiments other than those discussed above. This method of disclosure is not to be interpreted as reflecting an intention the present disclosure requires more features than are expressly recited in each claim. Rather, as the following claims reflect, inventive embodiments lie in less than all features of a single foregoing disclosed embodiment, configuration, or aspect. Thus, the following claims are hereby incorporated into this Detailed Description, with each claim standing on its own as a separate aspect of the present disclosure.

Moreover, though the description of the present disclosure has included description of one or more embodiments, configurations, or embodiments and certain variations and modifications, other variations, combinations, and modifications are within the scope of the present disclosure, e.g., as may be within the skill and knowledge of those in the art, after understanding the present disclosure. It is intended to obtain rights which include alternative embodiments, configurations, or embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A seizure detection system (100) comprising:
an input unit (104) configured to obtain electroencephalography (EEG) data and photoplethysmography (PPG) data to generate a first signal representing the EEG data and the PPG data;
a pre-processing unit (106) coupled to the input unit (104), and configured to, upon receipt of the first signal, pre-process the PPG data and the EEG data to generate a second signal;
first processing circuitry (108) coupled to the pre-processing unit (106), and configured to, upon receipt of the second signal, (i) generate at least three outputs and (ii) generate a combined output by combining the at least three outputs, wherein the first processing circuitry (108) further generates a third signal based on the combined output;
a post-processing unit (110) coupled to the first processing circuitry (108), and configured to, upon receipt of the third signal (i) post-process the combined output to generate an event score and (ii) compute one of, the EEG data and the PPG data to obtain one or more physiological parameters of a subject;
second processing circuitry (112) coupled to the post-processing unit (110), and configured to combine the event score and the one or more physiological parameters to generate a fourth signal representing a final output; and
an output unit (114) coupled to the second processing circuitry (112), and configured to, upon receipt of the fourth signal, predict a seizure based on the final output.

2. The seizure detection system (100) as claimed in claim 1, further comprising a database (102) that is coupled to the input unit (104) such that the database (102) stores the EEG data and PPG data, wherein the input unit (104) is configured to obtain the EEG data and the PPG data from the database (102).

3. The seizure detection system (100) as claimed in claim 1, wherein the input unit (104) comprises a plurality of sensors (116a, 116b) that are configured to sense EEG and PPG signals representing the EEG and the PPG data, respectively.

4. The seizure detection system (100) as claimed in claim 1, wherein, to pre-process the EEG data, the pre-processing unit (106) is configured to remove a plurality of artefacts from the EEG data.

5. The seizure detection system (100) as claimed in claim 1, wherein the output unit (114) comprising third processing circuitry (118) that is coupled to the second processing circuitry (112), and configured to (i) predict the seizure and (ii) determine, upon receipt of the fourth signal, phase of the seizure.

6. The seizure detection system (100) as claimed in claim 5, wherein the third processing circuitry (118) is further configured to generate an alert signal upon prediction of the seizure.

7. The seizure detection system (100) as claimed in claim 6, further comprising a notifier (120) that is configured to generate a notification pertaining to the seizure upon generation of the alert signal to notify at least one of, a care-giver, a health-personnel, and a health authority.

8. The seizure detection system (100) as claimed in claim 1, wherein the one or more physiological parameters comprises heart rate variability and blood pressure.

9. The seizure detection system (100) as claimed in claim 6, wherein the third processing circuitry (118) is further configured to generate a null signal, when at least one of (i) seizure events<30 seconds, (ii) seizure events<15 seconds, and (iii) merged seizure events with P (seizure)<82%, is met.

10. A method (300) for detecting seizure, the method (300) comprising:
obtaining (302), by way of an input unit (104), electroencephalography (EEG) data and photoplethysmography (PPG) data, to generate a first signal representing the EEG data and the PPG data;
pre-processing (304), by way of a pre-processing unit (106) that is coupled to the input unit (104), the EEG data, upon receipt of the first signal, to generate the second signal;
generating (306), by way of first processing circuitry (108) that is coupled to the pre-processing unit (106), upon receipt of the second signal, at least three outputs;
generating (308), by way of the first processing circuitry (108), a combined output by combining the at least three outputs to generate a third signal based on the combined output;
post-processing (310), by way of a post-processing unit (110) that is coupled to the first processing circuitry (108), upon receipt of the third signal, the combined output to generate an event score;
computing (312), by way of the post-processing unit (110), one of, the EEG data and the PPG data to obtain one or more physiological parameters of a subject;
combining (314), by way of second processing circuitry (112) that is coupled to the post-processing unit (110), the event score and the one or more physiological parameters to generate a fourth signal representing a final output; and
predicting (316), by way of an output unit (114) that is coupled to the second processing circuitry (112), upon receipt of the fourth signal, a seizure based on the final output.

11. The method (300) as claimed in claim 10, further comprising alerting (318), by way of a notifier (120) of the output unit (114), the subject about the seizure, wherein to alert, the output unit (114) is configured to generate an alert signal such that the notifier (120) is configured to generate a notification pertaining to the seizure, upon generation of the alert signal.
